(19) United States
(12) Patent Application Publication
(10) Pub. No.: US 2010/0273746 A1
(43) Pub. Date: Oct. 28, 2010

(54) PHARMACEUTICAL FORMULATIONS CONTAINING TOLPERISONE

(76) Inventors: Ottilia Balázs, Budapest (HU); Karoly Tihanyi, Budapest (HU); Katalin Hornok, Budapest (HU); Istvanné Kis-Varga, Kakucs (HU); Mária Virágh-Hadas, Budapest (HU)

Correspondence Address: HAHN & VOIGHT PLLC, 1012 14TH STREET, NW, SUITE 620, WASHINGTON, DC, 20005, US

(21) Appl. No.: 12/809,905

(22) PCT Filed: Dec. 18, 2008
(86) PCT No.: PCT/HU08/00153

§ 371 Date: Jun. 21, 2010

(30) Foreign Application Priority Data
Dec. 20, 2007 (HU) P0700828

(51) Int. Cl. A61K 31/60; A61K 31/4453; A61K 31/541; A61P 19/02; A61P 29/00

(52) U.S. Cl. 514/161; 514/317; 514/226.5

(57) ABSTRACT

The present invention relates to pharmaceutical formulation containing tolperisone or its pharmaceutically acceptable salts or tolperisone combined with a non-steroidal anti-inflammatory drug or their salts, gel forming macromolecule, solvent, and if required thickening agent, penetration enhancer and pH adjuvant or the mixture thereof. The invention also relates to the manufacturing process of the above mentioned pharmaceutical compositions, further the use of these formulations and the containers suitable for the dosage, which are dual compartment containers consisting of two separated chambers.

The invention relates to a novel pharmaceutical formulation comprising tolperisone or its pharmaceutically acceptable salts or tolperisone and non-steroidal anti-inflammatory drug or their pharmaceutically acceptable salts, gel forming macromolecule, solvent and if required thickening agent, penetration enhancer and pH adjuvant or a mixture of any thereof.

Furthermore, the present invention relates to a process for the preparation of this novel pharmaceutical formulations and the use of this novel pharmaceutical formulations for the treatment of musculoskeletal trauma (for instance sport injuries, bruises, dislocations), low back pain, back pain, rheumatoid arthritis, osteoarthritis and ankylosing spondylitis.

The increased level of prostaglandin and sodium channel expression play a significant role in the development of typical symptoms (inflammation, oedema, pain) of musculoskeletal inflammatory diseases. In the clinical practice non-steroidal anti-inflammatory drugs, which inhibit the synthesis of prostaglandin, are the first choice in the topical treatment of musculoskeletal disorders. In case of oral administration the non-steroidal anti-inflammatory drugs generate serious gastrointestinal (GI) side effects whose incidence and degree is proportional to the plasma concentration of the active ingredient. For that reasons the local application of non-steroidal anti-inflammatory drugs, as a possible therapeutic approach has a significant importance in case of high-risk patients (old people, patients with antecedent gastrointestinal problems). However, the therapeutic value of topically applied non-steroidal anti-inflammatory drugs is limited by the long lag time and relative short duration of antihyperalgesic activity.

The antihyperalgesic effect of sodium channel blocking agents (lidocain, mexiletin, tocainamid) is well known, these molecules are widely used as local anaesthetics because of their immediate action. Additionally, the gel containing 5% lidocain is proved to be effective for the symptomatic treatment of neurophatic pain.

Tolperisone (2-methyl-1-(4-methyl-phenyl)-3-(1-piperidinil)-1-propanon, see: HU Pat. 144,997) as a central muscle relaxant has been used in the clinical practice for more than 40 years. Besides its muscle relaxant activity the molecule also has antihyperalgesic effect, which has been demonstrated by experimental and human data. Due to its membrane stabilizer effect tolperisone inhibits the nerve conduction of primer afferent fibres, thereby blocking the mono and polysynaptic reflexes in the spinal cord. In addition, tolperisone inhibits the synaptic influx of calcium ions and presumably the efflux of neurotransmitter. The high potential pharmacological efficacy of the molecule is decreased considerably by low bioavailability and first pass metabolism caused by CYP2D6 and ketoreductases.

Tolperisone is preferable to be applied in the human therapy due to its advantageous side effect profile and safe administration. In contrast to other central muscle relaxant tolperisone has no sedative effect; 900 mg can be taken daily without significant adverse reaction. Albeit that many pharmaceutical preparations containing tolperisone are available on the world market, there is no data regarding topical dosage forms incorporating tolperisone in the literature. The lack of tolperisone topical preparation on the pharmaceutical market seems to be the result of the chemical instability of the compound, which complicates the formulation of transdermal compositions containing tolperisone. In aqueous solution tolperisone, as a Mannich-ketone decomposes to piperidine and vinyl ketone in hydrolytic reaction. This process is catalysed by temperature, light and hydroxide ions.

Providing a number of advantages transdermal application, which has gained ground more and more in modern clinical practice, can be used successfully for administration of drugs encountering first-pass metabolism, and molecules with a narrow therapeutic window or short half-life. In case of transdermal application aiming at a systematic exposition, drugs get equally and continuously into the blood circulation, the pharmacological effect can be easily ceased by removing the composition. Transdermal applications also offer opportunity for non-invasive local treatment. Using transdermal therapy high drug concentration can be achieved in the target tissue without raising systemic side effects; therefore this is a comfortable route of administration for patients and nursing staff alike.

Conventional gel formulations, which have a great importance among the semi-solid transdermal dosage forms, can incorporate a considerable amount of aqueous solvent, and contrary to hydrocarbon gels or lipogel systems they neither impede the physiological skin functions nor close the pores providing effective drug penetration trough the skin. Thanks to their softer consistency, hydrogel formulations can be spread easily, they have more aesthetic appearance than lipogel systems. Gel formation is often induced by alkaline pH or elevated temperature. In case of widely used synthetic macromolecules such as acrylate polymers, polymethacrylates (Carbopol gels) the gel formation is thought to occur after neutralization under alkaline condition, which limits the formulation of those molecules which tend to decompose at alkaline pH.

The wetting of semisynthetic macromolecules (cellulose derivates) is carried out by using water or aqueous solvent at 60-90° C. In many cases these systems also comprise plasticizer, which is often monohydric or dihydric alcohol containing a considerable amount of water. Based on high water content and special manufacturing conditions hydrogels are not suitable for formulation of molecules which are thermodynamic unstable or tend to decompose in hydrolytic reaction.

The WO 02/089849 discloses the above mentioned hydrogel systems. The composition comprises a local anaesthetic agent and a pharmaceutically acceptable, non liposomal carrier comprised of a monohydric alcohol, a penetration enhancer and hydrophilic or hydrophobic polymer or a combination thereof. Taking into consideration that these gels contain added water in any cases, Tolperisone must be unstable in these compositions. It is known from the literature that although there are compositions developed with the aim of achieving entirely water-free system, it did not work out because of residual water content of the applied excipients whose negative effect was neglected.

The U.S. Pat. No. 5,446,070 Patent Specification describes compositions with relatively high active ingredient content (1-70%). These transdermal formulations consist of a pharmaceutically acceptable solvent including a certain amount of plasticizer and a polysaccharide bioadhesive carrier in an amount from 20 to 34 weight percent. According to the examples polihydric alcohol is used as a solvent and the plasticizer is glycerine in any case. Although the manufacturing process is performed without water addition, the invention is not applicable to tolperisone formulation since neither the residual water content of the excipients nor the effect of the humidity absorbed adventitiously during the procedure are taken into account, moreover the manufacturing process needs high temperature (50° C.-130° C.). Additionally, in spite of the used technical term: ‘free of water’, one of the examples contains 85% water as a solvent.

The U.S. Pat. No. 5,719,197 describes a bioadhesive composition produced at elevated temperature (100° C.) without water addition. The scope of the invention is similar to the previous mentioned patent with a slight difference that this composition comprises mineral materials (for instance clay, bentonite, zinc oxide) additionally. Based on the afore-mentioned reasons this formulation is not suitable for tolperisone formulation either.

Organic acids or bases are often used practically to decrease the residual water content of inactive ingredients which contain less than 0.02 percent of water. In organic solvents these acids and bases are in-non ionised form. In the presence of water they will ionise proportionally to the amount of water resulting acidic or alkaline micro-environments which can assure long term stability of active ingredients. The applied alpha-hydroxiacids (abbreviated AHA), for instance ascorbic acid, citric acid, lactic acid, glycolic acid, malic acid, tartaric acid) enhance the hydration of stratum corneum thereby improve drug penetration through the skin.

The Japanese patent application No. 2000/143510 describes a composition for external use containing tolperisone as a penetration enhancer. According to the invention tolperisone is applied as an inactive ingredient in the pharmaceutical composition, there is no information on the own therapeutic effect of tolperisone. All dosage forms given in the concerning patent contain additional water in an amount of 40 weight percent, therefore considering the physicochemical properties of tolperisone these pharmaceutical compositions have no relevance in actual practice.

During the formulation of a transdermal preparation it has to be taken into account that the applied active ingredient can be incompatible with excipients or in case of combinations with other active ingredient. To avoid incompatibility, these pharmaceutical preparations can be packaged in dual compartment containers.

The aim of the present invention was to examine the anti-inflammatory and antihyperalgesic effects of pharmaceutical compositions containing tolperisone or tolperisone combined with non-steroidal anti-inflammatory drugs. Furthermore our aim was to develop a pharmaceutical preparation for external use, in which the gel formation does not need water addition and it is independent from temperature and pH conditions providing a promising approach to formulation of very sensitive tolperisone.

Our experiments were focused on developing water-free gel formulations, which contrary to conventional gel systems, contain high amount of organic solvent as a hydrophilic medium and their manufacturing process does not depend on temperature or pH conditions. Pharmaceutically and cosmetically acceptable organic solvents were used such as dimethyl sulfoxide, propylene glycol, diethyl ene glycol monoethyl ether (Transcutol®), propylene carbonate, polyethylene glycols with molecule weight ranged from 200 to 900 dalton or mixture thereof. To develop a water-free gel structure semisynthetic and synthetic macromolecules were applied, for instance cellulose derivates (Metolose®, Metocel®, Pharmacoat®) and poly(acrylic acid) derivates (Carbopol®, Pemulen®, Noveon®).

During our experiments surprisingly it was found that the applied solvents and their mixture also functioned as plasticizers, so the gel structure formation occurred without additional plasticizer or the necessity of changing the temperature or pH conditions of the system.

We have found that in gel compositions tolperisone showed appropriate stability even under accelerated test condition. According to our in-vivo experiments, locally administered tolperisone resulted in significant anti-inflammatory and analgesic effect in animal models that reliably mimic the symptoms of musculoskeletal inflammatory diseases.

During our further experiments surprisingly it was found that the combination of sodium channel blocking agent tolperisone and a prostaglandin synthesis inhibitor non-steroidal anti-inflammatory drug presented significant anti-inflammatory and analgesic effect.

According to our in-vivo experiments, local application of gel compositions containing tolperisone resulted in a highly significant dose dependent reduction of pain and inflammation in animal models. The composition containing 10% tolperisone has an anti-inflammatory effect comparable to transdermal gel formulations available on the market, which comprise propionic acid derivative non-steroidal anti-inflammatory drugs, such 2.5% ketoprofen or 5% ibuprofen. Tolperisone appears to be associated with a lower risk of adverse effects than non-steroidal anti-inflammatory drugs. The transdermal pharmaceutical formulations containing tolperisone and a non-steroidal anti-inflammatory drug show early emerging and long-lasting intensive effect.

The present invention relates to a novel pharmaceutical formulation comprising tolperisone or its pharmaceutically acceptable salts or tolperisone and non-steroidal anti-inflammatory drug or their pharmaceutically acceptable salts, gel forming macromolecule, solvent and if required thickening agent, penetration enhancer and pH adjuvant or a mixture of any thereof.

In one embodiment of the present invention the pharmaceutical composition contains tolperisone or preferably tolperisone hydrochlorid at a dose range of from 2.5 w/w % to 20 w/w % and a non-steroidal anti-inflammatory drug at a dose range from 2.5 w/w % to 20 w/w %.

According to the invention the pharmaceutical formulation preferably contains a non-steroidal anti-inflammatory drug selected from diclofenac, aceclofenac, naproxen, ibuprofen, indomethacin, piroxicam, flurbiprofen, ketoprofen, acetyl salicylic acid, sulindac, niflumic acid, metamizol, benzydamine, paracetamol and their pharmaceutically acceptable salts.

According to the invention the pharmaceutical formulation preferably contains the gel-forming agent selected from colloidal silicon dioxide, cellulose derivates, polyoxyalkylene and its derivates, acrylate polymer or a mixture of any thereof. The gel-forming agent is preferably selected from hydroxypropyl methyl cellulose ethers and poly(acrylic acid) derivates or a mixture of any thereof.

In further embodiment of the invention the solvent is selected from dimethyl-sulfoxide, diethylene-glycol-mono ethyl-ether, propylene-carbonate, polyethylene-glycol, pirrolidone or its derivate, N-substituted-alkyl-azacycloalkyl-2-ones derivate, dimethyl-formamide, acetamide, propylene-glycol or a mixture of any thereof. The solvent is preferably selected from dimethyl-sulfoxide, propylene-glycol, diethylene-glycol-mono ethyl-ether or a mixture of any thereof.

The thickening agent is selected from monohydric and polyhydric alcohols, polyethylene-glycol with molecular weight ranged from 80 to 20 000 dalton, propylene-glycol or a mixture of any thereof. The thickening agent is preferably propylene-glycol.

The penetration enhancer is selected from fatty acid, fatty acid ester, polyoxylglyceride, N-substituted alkyl-azacycloalkyl-2-ones derivate, menthol, terpene, essential oils, phospholipide, sulfoxide, amino-acid and its derivate, enzime or a mixture of any thereof. The penetration enhancer is preferably polyethylene glycol fatty acid ester.

The pH adjuvant is selected from alpha-hydroxy acids, dicarboxylic acid, aromatic acid, polyhydroxycarboxylic acid or a mixture of any thereof. The pH adjuvant is preferably selected from the group consisting of ascorbic acid, citric acid or tartaric acid.

The invention further relates to a process for the preparation of a pharmaceutical formulation by dissolving the pharmaceutically active agent or agents and pH adjuvant in a solvent under nitrogen atmosphere, dispersing the gel forming agent and other pharmaceutical excipients in the solution containing active ingredient.

The invention further relates to container for administration of the pharmaceutical formulation which is a dual compartment container consisting of two separated chambers.

The invention further relates to a pharmaceutical gel formulation for external use in medical therapy.

The invention further relates to use of tolperisone or its pharmaceutically acceptable salts or tolperisone combined with non-steroidal anti-inflammatory drugs for transdermal treatment of musculoskeletal trauma (for instance sport injuries, bruises, and dislocations), low back pain, back pain, rheumatoid arthritis, osteoarthritis and spondylitis anchylopoetica.

The pharmaceutical compositions according to our invention have the advantage of assuring stable gel formulations containing tolperisone and its pharmaceutically acceptable salts, which are susceptible to decompose in hydrolytic reaction. Water-free delivery systems are suitable for manufacturing transdermal preparations containing tolperisone and a non-steroidal anti-inflammatory drug with different physicochemical properties.

Tolperisone alone or in combination with a non-steroidal anti-inflammatory drug remains stable in the developed water-free delivery system.

The pharmaceutical compositions according to our invention have further advantages, since their microbiological conservation is easier than in case of hydrogel formulation due to the fact that high organic solvent content is not favourable for the growth of microbes. It has importance because preservatives used in hydrogel formulation can cause allergic reaction and at higher concentration they can even be toxic. The pharmaceutical compositions according to our invention have favourable aesthetic appearance and they are easy to remove with water.

During our experiment following methods were applied:

Two experiments were conducted to investigate the chemical stability of the pharmaceutical preparations:

a.) Chemical stability of pharmaceutical gel preparation manufactured according to Example 1 was investigated and compared to the pharmaceutical gel preparation, which had almost the same composition except that it contained purified water instead of dimethyl-sulfoxide.

During the stability test the gels were stored at room temperature (25° C.) and under accelerated condition (40° C., 75% RH). Chemical stability of the samples was evaluated using spectrophotometric quantification of piperidine, one of the degradation products of Tolperisone. It was extracted with chloroform; the optical density of piperidine was measured at 465 nm using Hitachi U-3010 Spectrophotometer. Prior to the measurement piperidine was reacted with 1,2 naphtokinon-4-sulfonic acid. The analytical method is linear (R=0.9943) between 10 μg and 200 μg amount of piperidine. The amount of degradation product was given in percentage of initial tolperisone content. In the tolperisone water-free gel formulation stored at room temperature 0.6% of piperidine was detected after 1 month, while in the tolperisone water containing gel formulation stored under the same condition 1.2% of degradation product was measured. In the tolperisone water-free gel formulation, which was stored under accelerated condition (40° C., 70% RH) approximately one order of magnitude lower quantity of degradation product (0.94%) was measured than in the tolperisone water containing gel (8.9%) stressed under the same condition. (See: Table 1)

TABLE 1

| | | Piperidine | Piperidine |
|---|---|---|---|
| | | (Tolperizone %) | (Tolperizone %) |
| | | Room temperature, | 40° C./70% RH, |
| Composition | | 1 month | 1 month |
| Tolperisone HCl | 5% | 0.6 | 0.94 |
| Metolose 60SH- | 1.3% | | |
| 4000 | | | |
| Citric acid | 3% | | |
| Dimethyl-sulfoxide | 70.7% | | |
| Propylene-glycol | 20% | | |
| Tolperisone HCl | 5% | 1.2 | 8.9 |
| Metolose 60SH- | 1.3% | | |
| 4000 | | | |
| Citric acid | 3% | | |
| Purified water | 70.7% | | |
| Propylene-glycol | 20% | | |

b.) Tolperisone gel formulations containing 2.5%, 5% and 15% active ingredient were prepared according to Example 20, 21 and 22, and stored under accelerated stress condition (50° C., 70% RH) to investigate their stability. The measurement was based on the HPLC determination of vinyl-keton (the other main degradation product of tolperisone) using the parameters given below:
Column: Symmetry C8 3.5 μm, 75×4.6 mm I.D.
Mobile phase: “A” buffer 25 mM KH₂PO₄ pH=3.0“B” Methanol
Gradient: 0 minute 60% “A”, 20 minutes 10% “A”, 21 minutes 60% “A”, 25 minutes 60% “A”
Solvent: buffer: Acetonitrile=70:30
Flow rate: 1 ml/min
Detection: 220 nm and 260 nm UV
Column temperature: 35° C.
Temperature of sample compartment: 4° C.

The amount of degradation product (vinyl-keton) was given in percentage of initial tolperisone content. The tested gel formulations, packaged in polyethylene/aluminium/polyethylene laminated tubes, were stressed at elevated temperature (50° C.) to examine the change of vinyl-keton during the storage (0 day, 2 days, 5 days, 7 days).

Based on the results of these experiments surprisingly it was found, that the degradation of heat sensitive tolperisone expressed in vinyl-keton did not exceed the amount of 0.5% even at elevated temperature, despite the fact that the active ingredient was present in dissolved form in the composition, thereby the active was more vulnerable to chemical degradation. (See: Table 2)

TABLE 2

Vinil-ketone (in % of tolperisone)

| Storage at 50° C. | Initial | 2 days | 5 days | 7 days |
|---|---|---|---|---|
| Example 20 | 0 | 0 | 0.197 | 0.220 |
| Example 21 | 0 | 0 | 0.182 | 0.239 |
| Example 22 | 0 | 0.153 | 0.346 | 0.464 |

The invention will now be disclosed in further details, which should be considered as illustrative and non limitative of the present invention.

The pharmaceutical compositions according to the present invention can contain one or more active ingredients. The pharmaceutical preparations can comprise tolperisone, eperisone, silperisone and their pharmaceutically acceptable salts. In combination with the aforementioned molecules the pharmaceutical compositions can also contain one or more active ingredients such as phenyl acetic acid, antranyl acid, indolpropionic acid, pyrasolone derivate, benzothiazine derivate, sulfonamide or other group of non-steroidal anti-inflammatory drugs or their salts.

The pharmaceutical compositions according to the present invention contain non-steroidal anti-inflammatory drug selected from the group consisting of acetylsalicylic acid, benzydamine, diclofenac, aceclofenac, naproxen, ibuprofen, indomethacin, piroxicam, flurbiprofen, ketoprofen, sulindac, niflumic acid, metamizol, paracetamol.

In one embodiment the gel-forming agent is selected from the group consisting of microcrystalline cellulose, methyl cellulose, carboxymethylcellulose, cellulose ether, sodium carboxymethylcellulose, hydroxypropyl methylcellulose (Pharmacoat®, Hypromellose®, Metolose®), hydroxyethyl cellulose, quaterner ammonium salt of hydroxy ethyl cellusose polimer reacted with epoxid substituted with trimethyl ammonium group, colloidal silicon dioxide, carboxy vinyl polymer: poly(acrylic acid) derivates (Carbopor®, Pemulen®, Noveon®).

In another embodiment the pharmaceutical composition may contain dimethyl-sulfoxide, diethylene-glycol-monoethyl-ether, propylene-carbonate, polyethylene-glycol of different molecule weights, propylene glycol, 2-pyrrolidone, N-(2-hydroxy-ethyl)-pyrrolidone, N-methylpyrrolidone, dodecyl azyl cycloheptan 2-one and other n-substituted-alkyl-azacycloalkyl-2-ones derivates (Azone®), dimethyl-formamide, acetamide or mixture thereof as a solvent.

In further embodiment the pharmaceutical composition according to the present invention may also contain thickening agents, cosolvents and penetration enhancers selected from the group consisting of monohydric alcohol having chain lengths of 2-22 carbons such as ethanol, propanole, isopropanole, butanole, hexanole, cetyl alcohol, stearyl alcohol, di- or polyhydric alcohol having chain lengths of 2-22 carbons such as propylene glycol, glycerine, trihydroxy hexane such as 1,2,6 hexanetriol, sorbitol, 1,3 butanediol, 2,3 butanediol, polyethylene-glycols with molecular weight ranged from 80 to 20 000 dalton.

The pharmaceutical formulations according to the present invention may contain a penetration enhancer selected from ethanol, benzylalcohol, isopropyl alcohol, propylene glycol, glycerine, 2-pyrrolidone, N-(2-hydroxy-ethyl)-pyrrolidone, N-methylpyrrolidone, dodecyl azyl cycloheptan 2-one and other n-substituted-alkyl-azacycloalkyl-2-ones derivates, dimethyl-formamide, acetamide, menthol, diethylene-glycol-monoethyl-ether, dimethyl-sulfoxide, oleic acid, polysorbate 20, d-alpha-tocopheryl-polyethylene glycol-1000 succinate (Vitamin E TPGS 1000), propylene glycol monolaurate (Lauroglycol® 90), propylene glycol monocaprylate (Capryol® 90), polioxyglycerides such as caprylocaproyl macrogolglycerides (Labrasol®), macrogolglycerol hydroxystearate (Cremophor® RH40) and mixture thereof.

The pH adjuvants used according to the present invention may suitably be any pharmaceutically acceptable organic acids and their salts presenting in liquid or solid form e.g. adipic acid, malic acid, ascorbic acid, benzoic acid, tartaric acid, succinic acid, citric acid, fumaric acid, phatlic acid, glycolic acid, maleic acid, oxalic acid, propionic acid, sebacic acid, salicylic acid, lactic acid, polyhydroxy carboxylic acid.

Ethylenediamine tetra acetic acid (EDTA) or its salts are generally used to adsorb leached metal ions, which are derived from the excipients of the pharmaceutical compositions or the surface of equipment, in complex. If required, the pharmaceutical composition according to the present invention may comprise EDTA at a level of 0.01% to 0.1% by weight.

The pharmaceutical formulations according to the present invention may contain any conventional, pharmaceutically acceptable preservatives in an amount of 0.01% to 0.5% by weight, e.g. methyl-, ethyl-, propyl- and butyl ester of para hydroxy-benzoic acid, propyl gallate, sorbic acid and its sodium and potassium salts, propionic acid and its calcium and sodium salts, bronopole (2-bromo-2-nitro-1,3-propanediol) and salicylanilides such as disbromosalicylanilide, tribromosalicylamilides (Cinaryl), 1-(3-chloroallyl)-3,5,7-triaza-1-azanid-adamantane chloride (Dowicil), hexachlorophene, sodium benzoate, citric acid, ethylene diamine tetraacetic acid and its alkali metal and alkaline earth metal salts, butyl hydroxyanisol, butyl hydroxytoluene, phenolic compounds such as chloro- and bromocresols and chloro- and bromo-oxylenols, quaternary ammonium compounds like benzalkonium chloride, aromatic alcohols such as phenylethyl alcohol, benzyl alcohol, chlorobutanol, quinoline derivatives such as iodochlorhydroxyquinolin, and the like.

The pharmaceutical compositions according to the present invention may contain air or other pharmaceutically and cosmetically acceptable gases emulsified in the liquid phase of the composition with the aim of foam forming.

The pharmaceutical compositions used for external application aiming at local or systemic effect can be prepared in a suitable dosage form e.g. solutions, suspensions, emulsions, transparent or opaque gels, transdermal delivery systems, semisolid formulations including ointments, pastes, creams, emulsions with semisolid- or solid internal phase, emulsions, gels and solid foams with semisolid or liquid internal phase.

The pharmaceutical compositions can be administered in any container which is suitable for external application. Regarding the organic solvent content, the compositions should be avoided contacting directly with metal containers. The pharmaceutical compositions can be filled into pharmaceutical grade glass-, plastic- or laminated metal containers and dual compartment containers consisting of two separated chambers (dual dispensers or dual tubes).

The following non-limiting examples provide typical formulations for compositions of the present invention.

The compositions of the invention can be obtained with a method comprising the following steps:

Tolperisone/tolperisone salt or tolperisone/tolperisone salt combined with non-steroidal anti-inflammatory drugs and organic acid are mixed continuously at 500 rpm using Velp Arex magnetic stirrer, at room temperature by purging the solution with nitrogen until the components are dissolved entirely in the solvent. The gel forming agent is dispersed in 50% of the active ingredient's solution by the use of IKA RW 20 DZM paddle mixer working at 60 rpm, then it is left to swell. The swelling may take as much 12 hours. After complete swelling is attained, the remained 50% of the active ingredient's solution is added in equal portions to the system by continuous mixing to allow the formation of gel structure. The other excipients such as preservative or fragrances are dissolved in the remained 50% of the active ingredient's solution. The duration of final homogenization is at least 30 minutes at 100 rpm. The order as the components were mixed can not be changed. The gel system prepared in this way is transparent, easy to spread, do not leave stain and easy to remove with water.

The amounts of components are given in weight percent in the following examples.

### EXAMPLE 1

| | |
|---|---|
| Tolperisone HCl | 5% |
| Metolose 60SH-4000 | 1.3% |
| Citric acid | 3% |
| Dimethyl sulfoxide | 70.7% |
| Propylene glycol | 20% |

### EXAMPLE 2

| | |
|---|---|
| Tolperisone HCl | 2.5% |
| Metolose 60SH-4000 | 1.3% |
| Citric acid | 3% |
| Dimethyl sulfoxide | 73.2% |
| Propylene glycol | 20% |

### EXAMPLE 3

| | |
|---|---|
| Tolperisone HCl | 10% |
| Metolose 60SH-4000 | 1.3% |
| Tartaric acid | 3% |
| Dimethyl sulfoxide | 65.7% |
| Propylene glycol | 20% |

### EXAMPLE 4

| | |
|---|---|
| Tolperisone HCl | 5% |
| Ibuprofen | 5% |
| Metolose 60SH-4000 | 1.3% |
| Citric acid | 3% |
| Dimethyl sulfoxide | 65.7% |
| Propylene glycol | 20% |

### EXAMPLE 5

| | |
|---|---|
| Tolperisone HCl | 10% |
| Pharmacoat 606 | 9% |
| Citric acid | 3% |
| Transcutol: Dimethyl sulfoxide 1:3.4 | 78% |

### EXAMPLE 6

| | |
|---|---|
| Tolperisone HCl | 2.5% |
| Ibuprofen | 2.5% |
| Pharmacoat 606 | 7.5% |
| Ascorbic acid | 2% |
| Transcutol: Dimethyl sulfoxide 1:3.4 | 65.5% |
| Propylene glycol | 20% |

### EXAMPLE 7

| | |
|---|---|
| Tolperisone HCl | 5% |
| Carbopol 980 | 1.65% |
| Ascorbic acid | 2% |
| Cremophor RH40 | 20% |
| Propylene glycol | 71.35% |

### EXAMPLE 8

| | |
|---|---|
| Tolperisone HCl | 10% |
| Metolose 60SH-4000 | 1.5% |
| Dimethyl sulfoxide | 63.5% |
| Tartaric acid | 5% |
| Propylene glycol | 20% |

### EXAMPLE 9

| | |
|---|---|
| Tolperisone HCl | 5% |
| Carbopol 980 | 2.1% |
| Vitamin E TPGS | 20% |
| Tartaric acid | 5% |
| Propylene glycol | 67.9% |

### EXAMPLE 10

| | |
|---|---|
| Tolperisone HCl | 5% |
| Carbopol 980 | 2% |
| Ibuprofen | 5% |
| Tartaric acid | 3% |
| Dimethyl sulfoxide | 63.95% |
| Propylene glycol | 21.05% |

### EXAMPLE 11

| | |
|---|---|
| Tolperisone HCl | 5% |
| Ketoprofen | 5% |
| Metolose 60SH-4000 | 2% |
| Ascorbic acid | 5% |
| Dimethyl sulfoxide | 63% |
| Propylene glycol | 20% |

### EXAMPLE 12

| | |
|---|---|
| Tolperisone HCl | 5% |
| Benzydamine | 5% |
| Metolose 90SH-100 | 8% |
| Tartaric acid | 2% |
| Dimethyl sulfoxide | 60% |
| Propylene glycol | 20% |

### EXAMPLE 13

| | |
|---|---|
| Tolperisone HCl | 10% |
| Carbopol 980 | 2% |
| Ascorbic acid | 3% |
| Dimethyl sulfoxide:Transcutol 1:3.4 | 30% |
| Propylene glycol | 55% |

### EXAMPLE 14

| | |
|---|---|
| Tolperisone HCl | 5% |
| Metamizol | 5% |
| Carbopol 980 | 2% |
| Ascorbic acid | 1% |
| Dimethyl sulfoxide | 47% |
| Propylene glycol | 40% |

### EXAMPLE 15

| | |
|---|---|
| Tolperisone HCl | 2.5% |
| Diclofenac Na | 1% |
| Carbopol 980 | 2.3% |
| Citric acid | 2% |
| Dimethyl sulfoxide:Transcutol 1:3.4 | 72.2% |
| Propylene glycol | 20% |

### EXAMPLE 16

| | |
|---|---|
| Tolperisone HCl | 2.5% |
| Niflumin acid | 2.5% |
| Carbopol 980 | 3% |
| Citric acid | 1% |
| Dimethyl sulfoxide:Transcutol 1:3.4 | 73% |
| Propylene glycol | 18% |

### EXAMPLE 17

| | |
|---|---|
| Tolperisone HCl | 5% |
| Paracetamol | 2.5% |
| Carbopol 980 | 3% |
| Ascorbic acid | 0.5% |
| Transcutol:Dimethyl sulfoxide 1:3.4 | 89% |

### EXAMPLE 18

| | |
|---|---|
| Tolperisone HCl | 17.25% |
| Carbopol 980 | 2% |
| Ascorbic acid | 3% |
| Propylene glycol | 77.75% |

### EXAMPLE 19

| | |
|---|---|
| Tolperisone | 2.5% |
| Aceclofenac | 2.5% |
| Carbopol 980 | 2% |
| Dimethyl sulfoxide | 58% |
| Propylene glycol | 35% |

### EXAMPLE 20

| | |
|---|---|
| Tolperisone | 2.5% |
| Tartaric acid | 3% |
| Carbopol 980 | 2% |
| Propylene glycol | 92.5% |

### EXAMPLE 21

| | |
|---|---|
| Tolperisone | 5% |
| Tartaric acid | 3% |
| Carbopol 980 | 2% |
| Propylene glycol | 90% |

### EXAMPLE 22

| | |
|---|---|
| Tolperisone | 15% |
| Ascorbic acid | 3% |
| Carbopol 980 | 2% |
| Propylene glycol | 80% |

### EXAMPLE 23

| | |
|---|---|
| Tolperisone | 7.5% |
| Flurbiprofen | 7.5% |
| Tartaric acid | 3% |
| Carbopol 980 | 2% |
| Propylene glycol | 80% |

### EXAMPLE 24

| | |
|---|---|
| Tolperisone | 7.5% |
| Acetyl salicylic acid | 7.5% |
| Ascorbic acid | 3% |
| Carbopol 980 | 2% |
| Propylene glycol | 80% |

### EXAMPLE 25

| | |
|---|---|
| Tolperisone HCl | 5.75% |
| Tartaric acid | 3% |
| Carbopol 980 | 2% |
| Propylene glycol | 59.25% |
| Transcutol | 20% |
| Labrasol | 10% |

### EXAMPLE 26

| | |
|---|---|
| Tolperisone HCl | 11.5% |
| Tartaric acid | 3% |
| Carbopol 980 | 2% |
| Propylene glycol | 53.5% |
| Transcutol | 20% |
| Labrasol | 10% |

### EXAMPLE 27

| | |
|---|---|
| Tolperisone HCl | 17.25% |
| Tartaric acid | 3% |
| Carbopol 980 | 2% |
| Propylene glycol | 47.75% |
| Transcutol | 20% |
| Labrasol | 10% |

### EXAMPLE 28

| | |
|---|---|
| Tolperisone | 5% |
| Metolose 60SH-4000 | 1.3% |
| Lactic acid | 3% |
| Dimethyl sulfoxide | 70.7% |
| Propylene glycol | 20% |

### EXAMPLE 29

| | |
|---|---|
| Tolperisone | 5% |
| Metolose 60SH-4000 | 1.3% |
| Ascorbic acid | 3% |
| Dimethyl sulfoxide | 70.7% |
| Propylene glycol | 20% |

### EXAMPLE 30

| | |
|---|---|
| Tolperisone | 10% |
| Metolose 60SH-4000 | 1.3% |
| Citric acid | 3% |
| Dimethyl sulfoxide | 65.7% |
| Propylene glycol | 20% |

### EXAMPLE 31

| | |
|---|---|
| Tolperisone | 5% |
| Metolose 60SH-4000 | 1.3% |
| Dimethyl sulfoxide | 73.7% |
| Propylene glycol | 20% |

### EXAMPLE 32

| | |
|---|---|
| Tolperisone | 2.5% |
| Ibuprofen | 2.5% |
| Metolose 60SH-4000 | 1.3% |
| Citric acid | 3% |
| Dimethyl sulfoxide | 70.7% |
| Propylene glycol | 20% |

### EXAMPLE 31

| | |
|---|---|
| Tolperisone | 5% |
| Ibuprofen | 5% |
| Metolose 60SH-4000 | 1.3% |
| Dimethyl sulfoxide | 68.7% |
| Propylene glycol | 20% |

### EXAMPLE 34

| | |
|---|---|
| Tolperisone | 2.5% |
| Ibuprofen | 2.5% |
| Metolose 60SH-4000 | 1.3% |
| Dimethyl sulfoxide | 73.7% |
| Propylene glycol | 20% |

### EXAMPLE 35

| | |
|---|---|
| Tolperisone | 5% |
| Ibuprofen | 2.5% |
| Metolose 60SH-4000 | 1.3% |
| Citric acid | 3% |
| Dimethyl sulfoxide | 68.2% |
| Propylene glycol | 20% |

### EXAMPLE 36

| | |
|---|---|
| Tolperisone | 2.5% |
| Ibuprofen | 5% |
| Metolose 60SH-4000 | 1.3% |
| Citric acid | 3% |
| Dimethyl sulfoxide | 68.2% |
| Propylene glycol | 20% |

The pharmacodynamic effect of the pharmaceutical compositions containing tolperisone or tolperisone salt(s) and tolperisone combined with non-steroidal anti-inflammatory drug was tested by applying the pharmacological methods are given below. During the experiments 28-30 g NMRI male mice and 220-250 g male Vistar rats were used. The number of the animals involved into the experiments can be readable in the figures.

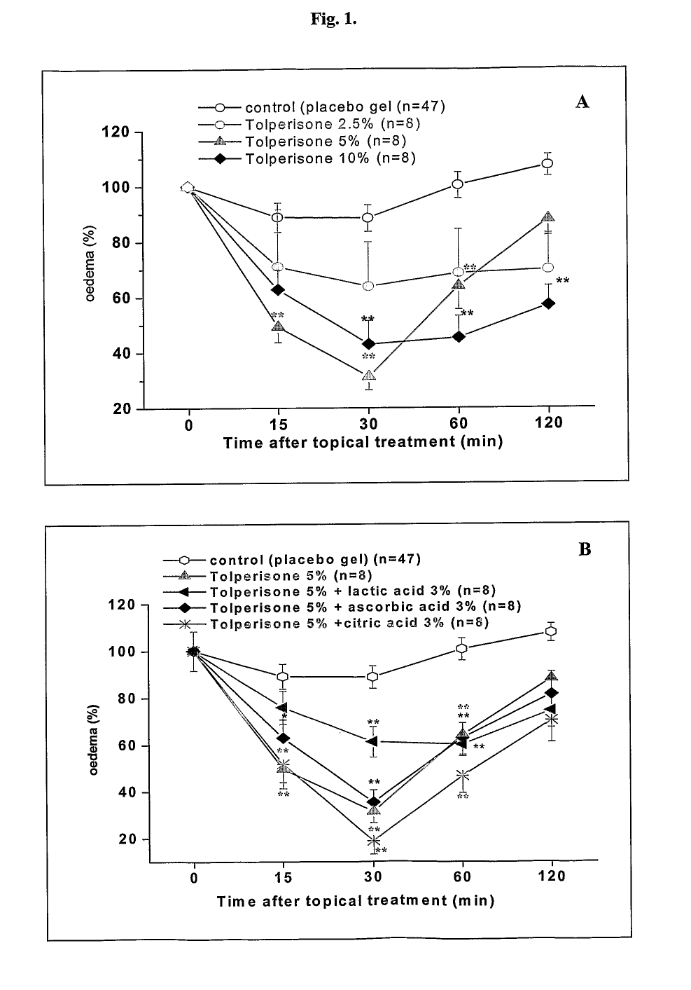

### 1. Complete Freund's Adjuvant (CFA) Induced Chronic Paw Oedema in Mice (Inflammation Test)—FIG. 1

The chronic inflammation of hind paw of NMRI mice was induced by subcutaneous injection of complete Freund's adjuvant Mycobacterium (CFA) 24 hours before the treatment. The tested compositions were applied by gentle rubbing on the sub-plantar region of the hind paw. Drug effects were expressed as the percentage reversal of the CFA-induced oedema (paw volume) increase versus pre-drug treatment (0 min) values. Mono component gel preparations containing 2.5-10% tolperisone (Example 1, 2, 30) have significant anti-inflammatory effect (FIG. 1A). The anti-inflammatory effect of 5% tolperisone gel is significantly enhanced by using 3% citric acid (FIG. 1B; Example 1, 28, 29, 31).

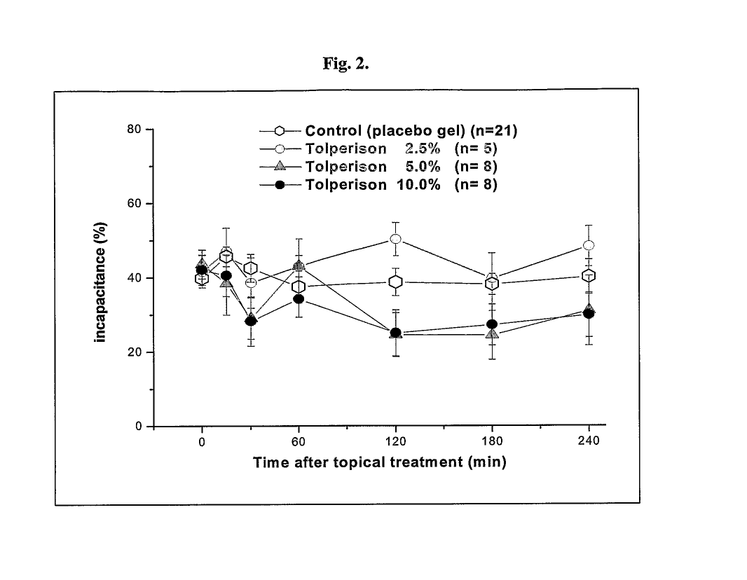

### 2. Complete Freund's Adjuvant (CFA) Induced Monoarthritis Model in Rats (Hyperalgesia Test) —FIG. 2

Male Wistar rats were injected with FCA into knee joint 72 hours before the treatment. The FCA injection induced chronic monoarthritis is characterized by severe inflammation and pain. The spontaneous pain is measured based on the decreased weight bearing capacity of the affected hind-limb. The incapacity (functional disability) is defined as the difference between the load exerted by the unaffected- and the affected limb, expressed in % of the total load exerted by both limbs. FIG. 2 illustrates the analgesic effect of gel preparations containing 2.5-10% tolperisone (Example 1, 2, 30).

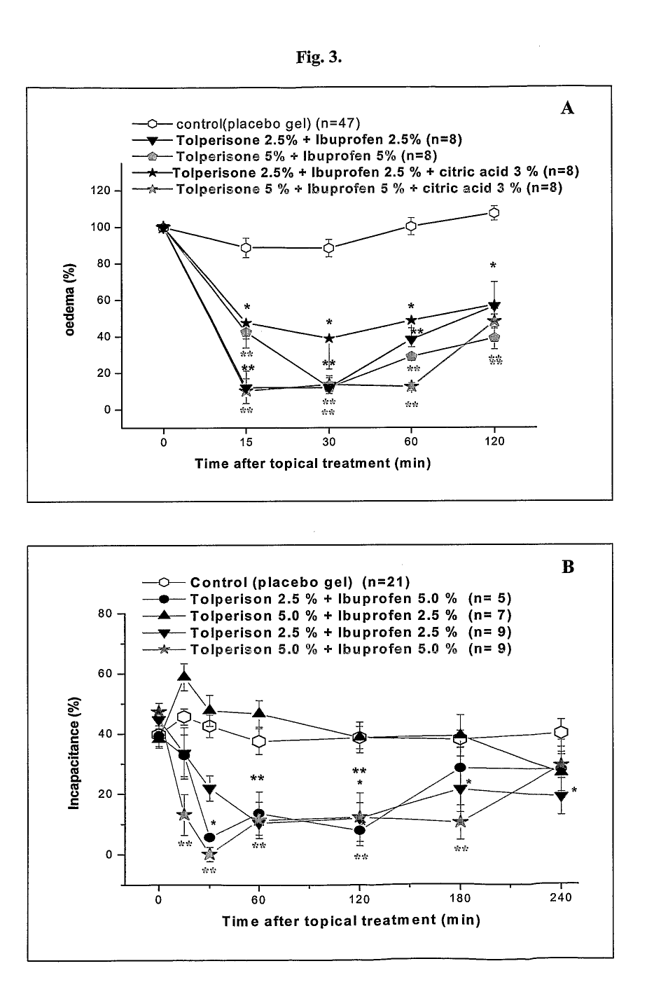

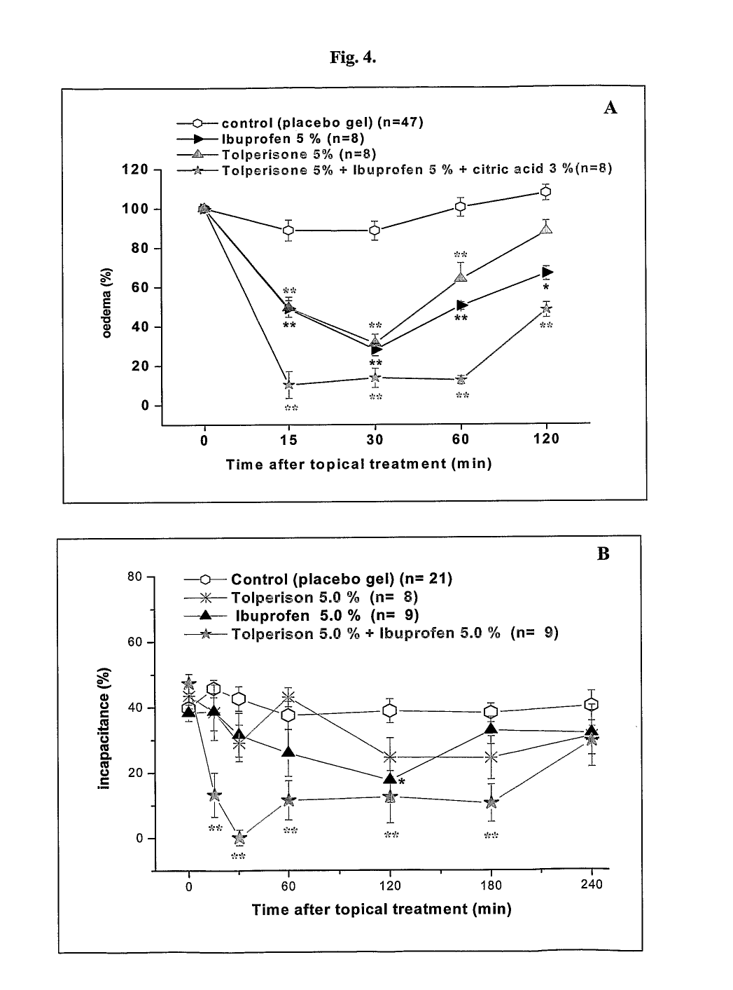

### 3. The Anti-Inflammatory and Analgesic Effects of Gel Combinations Containing Tolperisone and Ibuprofen in Different Concentrations and Ratio—FIGS. 3, 4.

In combination gels tolperisone (2.5% and 5%) and ibuprofen (2.5%, 5% and 10%) were studied. The weight ratios of active substances were 1:1, 1:2, and 2:1. The gel formulation containing 5% tolperisone combined with 5% ibuprofen and 3% citric acid (Example 4.) was proved to be the most effective composition.

This combination exhibited a rapid onset (15 min) of the pronounced anti-inflammatory and analgesic effect and the effects were sustained in time (180 min). The combination gel produced complete, 100% reversal of functional impairment at 30 minutes post-treatment (FIG. 3A, FIG. 3B; Example 4, 32, 33, 34, 35, 36).

The strong therapeutic effect of the composition containing 5% tolperisone and 5% ibuprofen is clearly a result of the supra-additive effect of the active ingredients in both pharmacological models (FIG. 4A, FIG. 4B; Example 1, 4, and composition based on Example 1 but containing 5% ibuprofen as active ingredient).

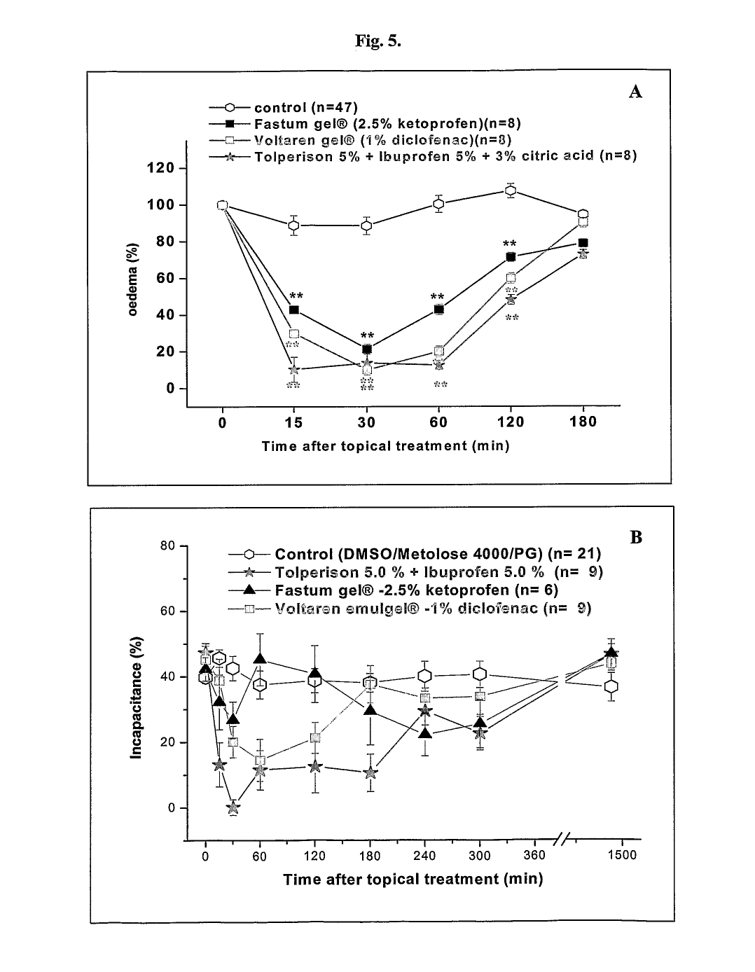

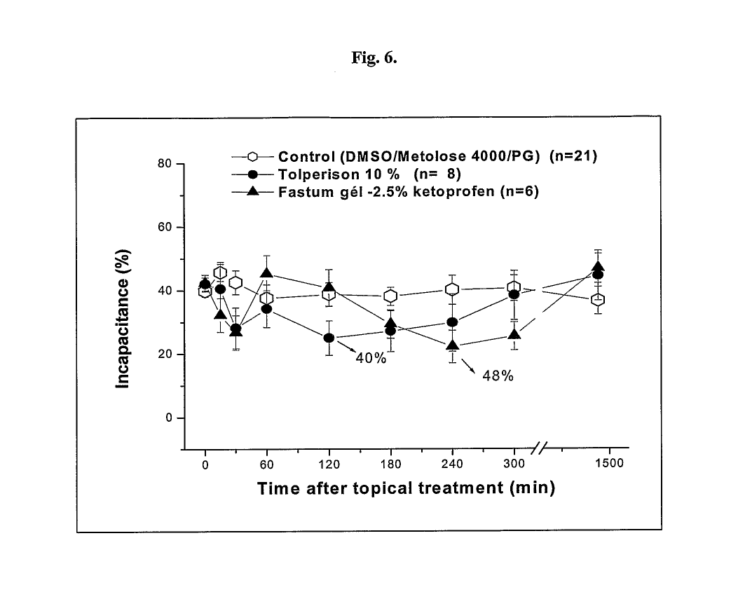

4. Comparative Anti-Inflammatory and Analgesic Effects of Tolperisone Gel Combinations with Marketed Topical Preparations Containing Non-Steroidal Anti-Inflammatory Drugs—FIGS. 5, 6.

The anti-inflammatory and pain relieving effects of the pharmaceutical compositions according to the present invention was compared with the marketed NSAID topical preparations: Fastum Gel® (2.5% ketoprofen Berlin-Chemie/Menarini group) and Voltaren Emulgel® (1% diclofenac Novartis/Consumer Health). According to FIGS. 5A and 5B the composition containing 5% tolperison, 5% ibuprofen and 3% citric acid (Example 4) has longer-lasting anti-inflammatory effect with similar potency than Fastum Gel® (2.5% ketoprofen) or Voltaren Emulgel® (1% diclofenac), but its analgesic effect is stronger, earlier emerging and longer lasting than the effect of the tested topical preparations available on the market. (FIG. 5A)

The pronounced analgesic effect of gel composition containing 5% tolperisone, 5% ibuprofen and 3% citric acid (Example 4) occurred 15 minutes after topical application and persisted for 3 hours. The combination gel produced complete, 100% reversal of functional impairment at 30 minutes post-treatment. (FIG. 5B)

The non-significant analgesic effect of Fastum Gel® appeared 3 hours post-treatment and could be measured for 3 hours. The slight maximum effect (48%) was detected 4 hours post-treatment. The analgesic effect produced by Voltaren Emulgel® started at 30 min post-treatment and was maintained for 3 hours. The maximum effect (68%) was detected 1 hour post-treatment. The anti-inflammatory and analgesic effect of gel preparation containing 10% tolperisone (Example 30) is similar to Fastum Gel®, but the analgesic effect is more potent and longer lasting than that of Fastum Gel®effect. (FIG. 6)

1. A water-free pharmaceutical formulation comprising tolperisone or its pharmaceutically acceptable salts or tolperisone and a non-steroidal anti-inflammatory drug or their pharmaceutically acceptable salts, a gel forming macromolecule, a solvent and if required a thickening agent, a penetration enhancer and a pH adjuvant or a mixture of any thereof.

2. A pharmaceutical formulation according to claim 1 which contains 2.5-20 w/w % tolperisone or tolperisone hydrochlorid.

3. A pharmaceutical formulation according to claim 1 which contains 2.5-20 w/w % nonsteroidal anti-inflammatory drug.

4. A pharmaceutical formulation according to claim 1 which contains a non-steroidal anti-inflammatory drug selected from diclofenac, aceclofenac, naproxen, ibuprofen, indomethacin, piroxicam, flurbiprofen, ketoprofen, acetyl salicylic acid, sulindac, niflumic acid, metamizol, benzydamine, paracetamol and their pharmaceutically acceptable salts.

5. A pharmaceutical formulation according to claim 1 wherein the gel-forming agent is selected from colloidal silicon dioxide, cellulose derivates, polyoxyalkylene and its derivates, acrylate polymer or a mixture of any thereof.

6. A pharmaceutical formulation according to claim 1 wherein the gel-forming agent is selected from hydroxypropyl methyl cellulose ethers and poly(acrylic acid) derivates or a mixture of any thereof.

7. A pharmaceutical formulation according to claim 1 wherein the solvent is selected from dimethyl-sulfoxide, diethylene-glycol-monoethyl-ether, propylene-carbonate, polyethylene-glycol, pirrolidone or its derivate, N-substituted-alkyl-azacycloalkyl-2-ones derivate, dimethyl-formamide, acetamide, propylene-glycol or a mixture of any thereof.

8. A pharmaceutical formulations according to claim 7 wherein the solvent is selected from dimethyl-sulfoxide, propylene-glycol, diethylene-glycol-monoethyl-ether or a mixture of any thereof.

9. A pharmaceutical formulation according to claim 1 wherein the thickening agent is selected from monohydric and polyhydric alcohols, polyethylene-glycol with molecular weight ranged from 80 to 20 000 dalton, propylene-glycol or a mixture of any thereof.

10. A pharmaceutical formulation according to claim 9 wherein the thickening agent is propylene-glycol.

11. A pharmaceutical formulation according to claim 1 which contains the penetration enhancer selected from fatty acid, fatty acid ester, polyoxylglyceride, N-substituted alkyl-azacycloalkyl-2-ones derivate, menthol, terpene, essential oils, phospholipide, sulfoxide, amino-acid and its derivate, enzime or a mixture of any thereof.

12. A pharmaceutical formulation according to claim 11 wherein the penetration enhancer is polyethylene glycol fatty acid ester.

13. A pharmaceutical formulation according to claim 1 wherein the pH adjuvant is selected from alpha-hydroxy acids, dicarboxylic acid, aromatic acid, polyhydroxycarboxylic acid or a mixture of any thereof.

14. A pharmaceutical formulation according to claim 13 wherein the pH adjuvant is selected from the group consisting of ascorbic acid, citric acid or tartaric acid.

15. A process for the preparation of a pharmaceutical formulation, as defined in claim 1, characterized by that dissolving the pharmaceutically active agent or agents and pH adjuvant in a solvent under nitrogen atmosphere, dispersing the gel forming agent and other pharmaceutical excipients in the solution containing active ingredient.

16. (canceled)

17. (canceled)

18. A method for transdermal treatment of musculoskeletal trauma (for instance sport injuries, bruises, and dislocations), low back pain, back pain, rheumatoid arthritis, osteoarthritis and spondylitis anchylopoetica, said method comprising external administration of tolperisone or its pharmaceutically acceptable salts or tolperisone combined with non-steroidal anti-inflammatory drugs.

19. The method of claim 18, further comprising filling a dual compartment container consisting of two separated chambers with the pharmaceutical formulation.
